(19) United States
(12) Patent Application Publication
(10) Pub. No.: US 2026/0114791 A1
(43) Pub. Date: Apr. 30, 2026

(54) NEUROLOGICAL STATE EVALUATION SYSTEM, NEUROLOGICAL STATE EVALUATION METHOD, AND NEUROLOGICAL STATE EVALUATION PROGRAM

(71) Applicant: Mecara LLC, Chiyoda-ku, Tokyo (JP)

(72) Inventors: Hiromi KAWAMATA, Chiyoda-ku, Tokyo (JP); Junichiro KOJOU, Chiyoda-ku, Tokyo (JP); Mitsuhiro MAEDA, Chiyoda-ku, Tokyo (JP); Hiromi YAMAGUCHI, Chiyoda-ku, Tokyo (JP)

(21) Appl. No.: 19/157,007

(22) PCT Filed: Jan. 24, 2024
(86) PCT No.: PCT/JP2024/001989

§ 371 Date: Aug. 15, 2025

(30) Foreign Application Priority Data
Feb. 17, 2023 (JP) 2023-023526

(51) Int. Cl. A61B 5/00; A61B 3/00; A61B 3/11; A61B 5/16

(52) U.S. Cl. CPC A61B 5/4035; A61B 3/0008; A61B 3/0025; A61B 3/0041; A61B 3/112; A61B 5/165; A61B 5/7235; A61B 5/7271; A61B 5/742; A61B 3/0033; A61B 5/7475

(57) ABSTRACT

A neurological state of a subject is accurately evaluated, and an evaluation result suitable for the subject is provided. A pupil index calculation unit generates waveform data of a pupil area based on a moving image in which a pupil of the subject is captured with a distortion-corrected lens, and then calculates a pupil index. The neurological state estimation unit estimates a sympathetic neural age and a parasympathetic neural age, which are neurological states for each of the left and right pupils of the subject, based on the pupil index. A comment generation unit evaluates the neurological state of the subject from the estimated neurological state, and the improvement measure generation unit generates advice suitable for the subject based on the evaluation result. A display unit displays the evaluation result of the neurological state and the advice in a neurological state evaluation system.

### TECHNICAL FIELD

The present invention relates to a neurological state evaluation system, a neurological state evaluation method, and a neurological state evaluation program.

### BACKGROUND ART

In recent years, the increase in individuals developing mental disorders such as diseases caused by stress, depression, and anxiety disorders has become a social problem. Judgment by a specialist who performs medical examinations is prone to subjectivity, and even a skilled physician finds it difficult to always accurately determine the mental and physical state of a patient. Against this background, the need to objectively evaluate the mental and physical state is increasing.

Patent Literature 1 describes a system that determines the mental and physical state of a subject based on temporal dilation and constriction data of the pupil in response to stimulus light. The system for determining the mental and physical state determines that, when the pupil size of the subject 5 seconds after the irradiation of stimulus light is larger than the pupil size at the moment of irradiation of the stimulus light, the subject is in a state where the sympathetic nervous system is dominant over the parasympathetic nervous system.

Patent Literature 2 describes a brain function examination method for examining the activity level of the autonomic nervous system, the presence or absence of dementia, and Alzheimer's disease, based on a discrimination index obtained by performing multivariate computation on various pupil indices derived by detecting the size of the pupil of the subject. Then, the brain function examination method uses, as pupil indices, two or more indices selected from among latency, pupil constriction time, pupil dilation time, initial pupil diameter, amount of pupil constriction, pupil constriction rate, pupil constriction velocity, maximum pupil constriction velocity, pupil dilation velocity, maximum pupil dilation velocity, pupil constriction acceleration, maximum pupil constriction acceleration, time to reach maximum pupil constriction velocity, time to reach maximum pupil dilation velocity, and time to reach maximum pupil constriction acceleration.

### CITATION LIST

### Patent Literatures

Patent Literature 1: Japanese Patent Application Laid-Open No. 2020-116312

Patent Literature 2: Japanese Patent Application Laid-Open No. 2002-34920

### SUMMARY OF INVENTION

### Technical Problem

The size and movement of the pupil are said to change according to age and to change before and after procedures or treatments. Until now, systems that determine the mental and physical state based on pupil data have merely determined the superiority between the sympathetic nervous system and the parasympathetic nervous system, or whether brain function is normal or not, and it is difficult to say that the systems have determined the mental and physical state accurately and in detail based on individual information such as the age of the subject. Therefore, it has not been easy to present optimal advice personalized to the subject.

The present invention has been conceived in view of such circumstances, and the object thereof is to estimate the neurological state of the subject more accurately and to provide an evaluation result suitable for the subject.

### Solution to Problem

The invention according to Claim 1 is a neurological state evaluation system including: a light irradiation unit configured to irradiate a pupil of a subject with light; an acquisition unit configured to acquire moving image data in which the pupil of the subject is captured; a calculation unit configured to calculate a pupil index from the moving image data; a state evaluation unit configured to evaluate a sympathetic neural age and a parasympathetic neural age of the subject based on the pupil index; and a display unit configured to display the sympathetic neural age and the parasympathetic neural age.

The invention according to Claim 2 is the neurological state evaluation system according to Claim 1, in which the state evaluation unit evaluates left and right sympathetic neural ages and parasympathetic neural ages based on left and right pupil indices of the subject, and the display unit displays each of the left and right sympathetic neural ages and parasympathetic neural ages.

The invention according to Claim 3 is the neurological state evaluation system according to Claim 1, in which the acquisition unit acquires the moving image data captured by a distortion-corrected lens, and the calculation unit calculates the pupil index by counting pixels related to the pupil in the captured moving image data.

The invention according to Claim 4 is the neurological state evaluation system according to Claim 1, in which the display unit displays a plurality of the pupil indices and a plurality of the neurological states at different time points or displays a change in the pupil indices or neurological states at different time points.

The invention according to Claim 5 is the neurological state evaluation system according to any one of Claims 1 to 4, further including an advice generation unit configured to generate advice to the subject based on the sympathetic neural age and the parasympathetic neural age, in which the display unit displays the advice.

### Advantageous Effect of Invention

According to the present invention, it is possible to estimate the neurological state of the subject accurately, and to provide an evaluation result suitable for the subject.

### BRIEF DESCRIPTION OF DRAWINGS

FIG. 1 is a diagram illustrating an overall configuration of an autonomic nervous system state determination system to which the present embodiment is applied.

FIG. 2 is a diagram illustrating a pupil diameter measurement device.

FIG. 3 is a diagram illustrating a functional configuration of a server.

FIG. 4 is a diagram describing parameters related to the pupil.

FIG. 5 is a diagram illustrating a functional configuration of a terminal device.

FIG. 6A is a diagram illustrating an input screen on a terminal device side of a neurological state evaluation system.

FIG. 6B is a diagram illustrating a screen for receiving a measurement start on the terminal device side of the neurological state evaluation system.

FIG. 7 is a diagram illustrating a measurement result display screen of the terminal device.

FIG. 8 is a diagram illustrating an advice display screen of the terminal device.

FIG. 9 is a flowchart illustrating a flow of processing of the terminal device.

FIG. 10 is a flowchart illustrating a flow of processing of the server.

FIG. 11 is a flowchart illustrating a flow of processing of the terminal device.

FIG. 12(A) is a diagram describing a pincushion distortion caused by a distortion lens, (B) is a diagram describing a barrel distortion caused by a distortion lens, (C) is a captured image in which a barrel distortion has occurred, and (D) is an image captured by a distortion-corrected lens.

FIG. 13 is a diagram illustrating a measurement result display screen of the terminal device.

### DESCRIPTION OF EMBODIMENTS

### Overall Configuration of Neurological state Evaluation System

Hereinafter, embodiments of the present invention will be described in detail with reference to the accompanying drawings.

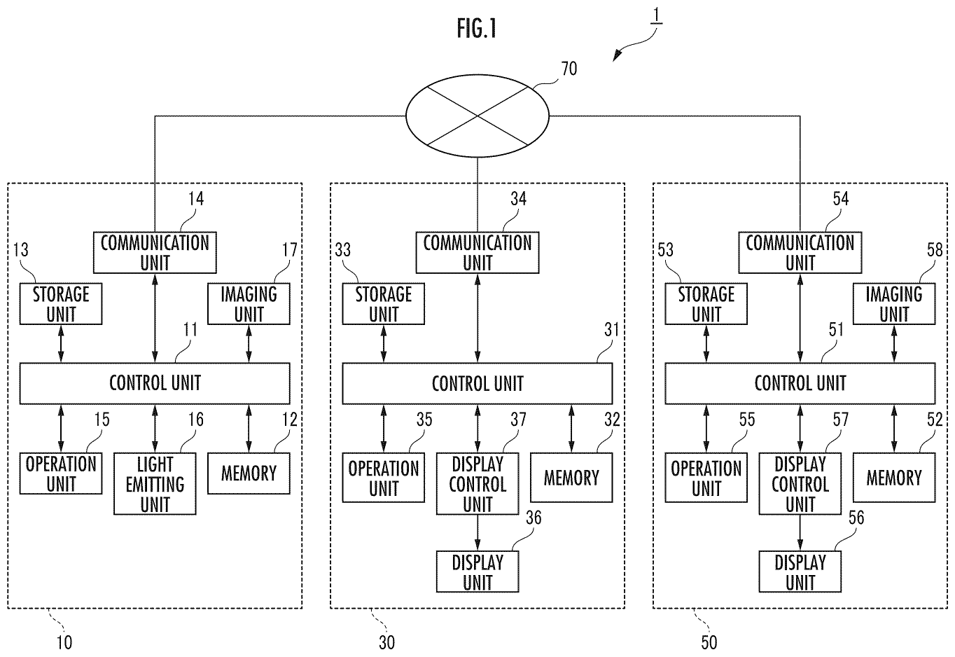

FIG. 1 is a diagram illustrating a hardware configuration of a neurological state evaluation system to which the present embodiment is applied.

A neurological state evaluation system 1 is a system in which a pupil meter 10 that measures a pupil of a subject, a server 30 that evaluates a neurological state of the subject, and a terminal device 50 that displays the neurological state of the subject are connected via a network 70. The neurological state evaluation system 1 exemplified in the present embodiment is a system that evaluates a neurological state of a subject based on moving image data in which a pupil of the subject is captured, and provides an evaluation result or advice to the subject or a user. As an example thereof, there is a system that analyzes and evaluates moving image data in which pupils of a subject are captured and provides information for improving an action of the subject.

The pupil meter 10 is configured as, for example, a dedicated measurement device that measures a pupil diameter. The pupil meter 10 includes a control unit 11 that is a processor (central processing unit (CPU)) controlling the entire pupil meter, a memory 12 such as a random access memory (RAM) used as a work area in computation, and a storage unit 13 that is a storage device such as a hard disk drive (HDD) or a semiconductor memory used for storing a program, various setting data, and the like. In addition, the pupil meter 10 includes a communication unit 14 that transmits and receives data, such as moving image data, via the network 70. Further, the pupil meter 10 includes: an operation unit 15 such as a measurement start button for receiving an input operation from a subject on the pupil meter 10 side; a light emitting unit 16 that irradiates the subject with light; and an imaging unit 17 that can capture the movement of the pupil of the subject as moving image data.

The server 30 is composed of a computer device such as a workstation, a desktop PC, or a notebook PC. The server 30 includes a control unit 31 that is a processor (central processing unit (CPU)) controlling the entire device, a memory 32 such as a random access memory (RAM) used as a work area in computation, and a storage unit 33 that is a storage device such as a hard disk drive (HDD) or a semiconductor memory used for storing a program, various setting data, and the like. In addition, a communication unit 34 that transmits and receives data via the network 70 is provided. Furthermore, an operation unit 35 that receives input operations from a user on the server 30 side, such as a keyboard, a pointing device, or a touch panel; a display unit 36 including a liquid crystal display that displays images, text information or the like to the server user; and a display control unit 37 that controls the display unit 36, are provided.

The terminal device 50 is configured by a computer terminal device such as a smartphone or a tablet. The terminal device 50 includes a control unit 51 that is a processor (central processing unit (CPU)) controlling the entire device, a memory 52 such as a random access memory (RAM) used as a work area in computation, and a storage unit 53 that is a storage device such as a hard disk drive (HDD) or a semiconductor memory used for storing a program, various setting data, and the like.

In addition, the terminal device 50 includes a communication unit 54 that transmits and receives data such as the moving image data and the evaluation result of the neurological state of the subject via the network 70. Furthermore, an operation unit 55 that receives input operations from a subject on the terminal device 50 side, such as a touch panel, a keyboard, or a pointing device; a display unit 56 including a liquid crystal display that displays images, text information or the like to the subject; and a display control unit 57 that controls the display unit 56. Further, the terminal device 50 includes an imaging unit 58 that can capture a moving image of the subject.

The CPU used in the pupil meter 10, the server 30, the terminal device 50, and the like or the CPU used in various devices connected to the pupil meter 10, the server 30, the terminal device 50, and the like constitutes “one or a plurality of processors” in the present application and implements various functions in the present embodiment.

In addition, various configurations of the pupil meter 10, the server 30, and the terminal device 50 illustrated in FIG. 1 do not necessarily have the same housing, and there is an aspect in which the pupil meter 10, the server 30, and the terminal device 50 are understood as a system. In a case where the main body device and the housing are different, the main body device and the housing are connected by wired or wireless.

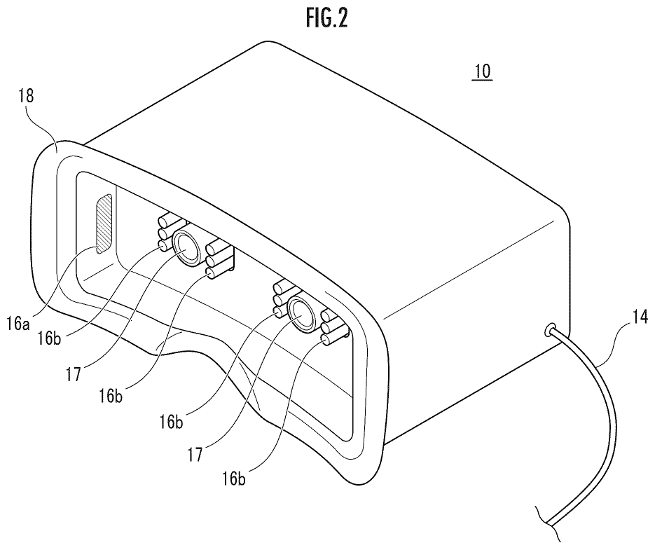

FIG. 2 is a conceptual diagram illustrating a functional configuration of the pupil meter. The pupil meter 10 includes: the communication unit 14 as wired or wireless communication means such as a communication cable; the light emitting unit 16 that irradiates the pupil of a subject with light; the imaging unit 17 that captures a moving image of the pupil of the subject; and a hood 18 that blocks light from the outside. The light emitting unit 16 includes a visible light emitting unit 16a and an infrared light emitting unit 16b. When the pupil meter 10 receives an input of a start of measurement from the server 30 or the terminal device 50 via the communication unit 14, the visible light emitting units 16a (only the left side is illustrated in FIG. 2) disposed on each of both the left and right sides inside the pupil meter housing irradiate the pupil of the subject with visible light for approximately 0.5 seconds in a direction in which the pupil is positioned. The photo stimulus that causes changes in the pupil is applied by irradiation with visible light, and furthermore, the infrared light emitting unit 16b irradiates the pupil with near-infrared light with a wavelength of 850 nm or more. The irradiation of the near-infrared light with a wavelength of 850 nm or more does not affect the change in pupil, but the pupil can be clearly and reliably captured by the irradiation with the near-infrared light. The image sensor constituting a part of the imaging unit 17 may be a complementary metal oxide semiconductor (CMOS) sensor capable of capturing from an infrared region to visible light. A camera lens constituting a part of the imaging unit 17 is disposed at a position where the pupil is easily captured within an interior covered by a hood. The captured moving image data is transmitted to the server 30 or the terminal device 50 via the communication unit 14. Although not illustrated, a storage unit that stores data of the captured moving image and the like, and a data processing unit that performs compression processing on the captured moving image data, may be provided.

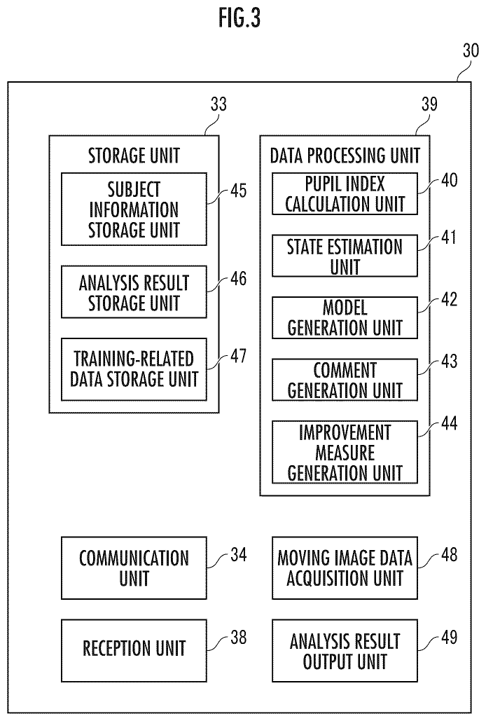

FIG. 3 is a diagram illustrating a functional configuration of the server. The server 30 includes the storage unit 33 that stores various types of data, the communication unit 34 that transmits and receives data such as the moving image and the evaluation result of the neurological state, a reception unit 38 that receives various types of input data, and a data processing unit 39 that analyzes the acquired moving image data. In the present specification, “analysis” includes not only analyzing information but also considering improvement measures and the like for improving a state determined from the analyzed result. Further, a moving image data acquisition unit 48 that acquires moving image data of the subject via the communication unit 34. In addition, an analysis result output unit 49 that outputs the analysis result to the display unit 56 of the terminal device 50 of the subject is provided. The “analysis result” may include advice, countermeasures, an improvement menu, and the like for improving the state determined from the analyzed result, in addition to the result of analyzing the information.

The storage unit 33 includes a subject information storage unit 45 that stores subject information, an analysis result storage unit 46 that stores an analysis result, and a training-related data storage unit 47 that stores a dataset for training and trained parameters.

The subject information stored in the subject information storage unit 45 has an aspect in which the subject information is acquired from the terminal device 50 and stored each time the analysis is performed, and an aspect in which the subject information is stored in advance together with identification information for identifying the subject. The “subject information” is information on an analysis target related to the subject, such as identification information of the subject, physical information such as height and weight, age, gender, a mental and physical state of the subject, and an examination history, which are the analysis target.

The analysis result storage unit 46 stores the data of the analysis result by the data processing unit 39 in association with the subject information stored in the subject information storage unit 45.

The training-related data storage unit 47 stores not only the pupil index of the subject, the actual neurological state of the subject, the actual age of the subject, and the like but also the trained parameters and the like. Further, the training-related data storage unit 47 may be a storage unit that stores the moving image data itself before the generation of the training data, the thinned-out moving image data, the moving image data in the middle of the analysis, and the compressed moving image data as well.

The data processing unit 39 includes: a pupil index calculation unit 40 that calculates an index related to the pupil; a state estimation unit 41 that estimates a neurological state of a subject; a model generation unit 42 that generates an estimation model; a comment generation unit 43 that generates a comment regarding the evaluation of the neurological state of the subject; and an improvement measure generation unit 44 that generates advice.

The pupil index calculation unit 40 calculates various parameters related to the pupil based on the moving image data of the pupil of the subject transmitted from the pupil meter 10. Specific pupil parameters will be described later.

The state estimation unit 41 estimates a neurological state of the subject based on a parameter (pupil index) related to the pupil. The inference model that estimates the neurological state of the subject may be a linear regression equation, a non-linear regression equation, or a neural network model. Further, the determination model may be a determination model using a decision tree, a support vector machine, or the like. The estimated data of the neurological state of the subject is transmitted to the terminal device 50 via the communication unit 34.

In the present embodiment, the “neurological state” refers to a mental and physical state of the subject or a brain function state caused by the activity of the nerves such as the autonomic nervous system and the cranial nerves. In addition, the neurological state is used as a concept including a physical state related to the nerves. For example, it is generally said that pain is likely to be felt when the sympathetic nervous system is dominant, and since pain is sometimes felt and sometimes not felt due to the activity of the autonomic nervous system, “pain” of the body is also included in the neurological state. In addition, even in a case where the subject does not feel pain in the subjective evaluation, a waveform pattern similar to pain may be detected from the measurement result of the pupil meter. Even such unconscious pain is also interpreted as being included in the “neurological state”in the present embodiment.

Further, the function of the autonomic nervous system peaks around 20 years old, and the neural function decreases with aging. The “neural age”, which indicates to which age's neural function level the subject corresponds based on the standard functional level by age, is also included in the “neurological state”. For example, the state estimation unit 41 estimates the neural level of the subject from a regression equation of an exponential function based on several pupil indices, and specifies the age corresponding to the level to calculate the neural age.

Further, the state estimation unit 41 evaluates the neurological state of the subject by comparing the estimated neurological state with an actual age of the subject. That is, in a case where the calculated neural age of the subject is 40 years old, and in a case where the actual age of the subject is 45 years old, the state estimation unit 41 evaluates the neurological state as being 5 years younger than the actual age in terms of neural age.

In the present example, since evaluating the neurological state includes estimating the neurological state, there is a case where the expression “evaluating the neurological state” is used even when only the neural age is simply estimated.

In addition, the state estimation unit 41 can also evaluate the neurological state by determining that the neurological state is in a “stress” or “tension” state based on pupil indices and the like related to the sympathetic nervous function or the parasympathetic nervous function.

The model generation unit 42 determines the parameters of the estimation model by performing machine learning based on the training data stored in the training-related data storage unit 47. Here, the machine learning refers to determining the parameters of a model such that the estimation error of the model, which estimates the dependent variable using the independent variables as input, becomes small based on a dataset of independent variables and dependent variables. Therefore, in a case where the estimation model is a regression equation, the machine learning refers to calculating a coefficient of the regression equation, and in a case where the estimation model is a neural network, the machine learning means determining a coefficient of a neuron. The determined model parameters are stored in the training-related data storage unit 47.

The comment generation unit 43 generates a comment describing the current situation of the neurological state based on the neurological state estimated by the generated estimation model. When generating the content of the comment, it is possible to perform highly accurate evaluation that matches the individual by generating the content of the comment in consideration of the subject information such as age in addition to the estimated neurological state. For example, the comment generation unit 43 creates a comment that evaluates the neurological state of the subject as having a younger neural age when the neural age is younger than the actual age, by comparing the age estimated from the pupil indices with the actual age of the subject. In the present example, “evaluating” the neurological state of the subject includes not only simply estimating the neurological state of the subject but also determining the neurological state of the subject including the estimated result. Therefore, diagnosing the neurological state in consideration of the comparison between the estimated neural age and the actual age of the subject also corresponds to “evaluation”.

The improvement measure generation unit 44 generates advice on improvement of working time or lifestyle or treatment of an injury based on the evaluation result of the neurological state. The improvement measure generation unit 44 may generate the advice by also taking into account the self-reported content input by the subject. For example, in a case where an evaluation result such as a spike-shaped waveform indicating pain appears in a waveform of a time-series change in pupil area from the evaluation result of the neurological state even though it is self-reported that there is no pain, the improvement measure generation unit 44 generates advice to receive medical treatment by a doctor because there is pain without subjective symptoms. The generated data of the comment or the advice is transmitted to the terminal device 50 via the communication unit 34.

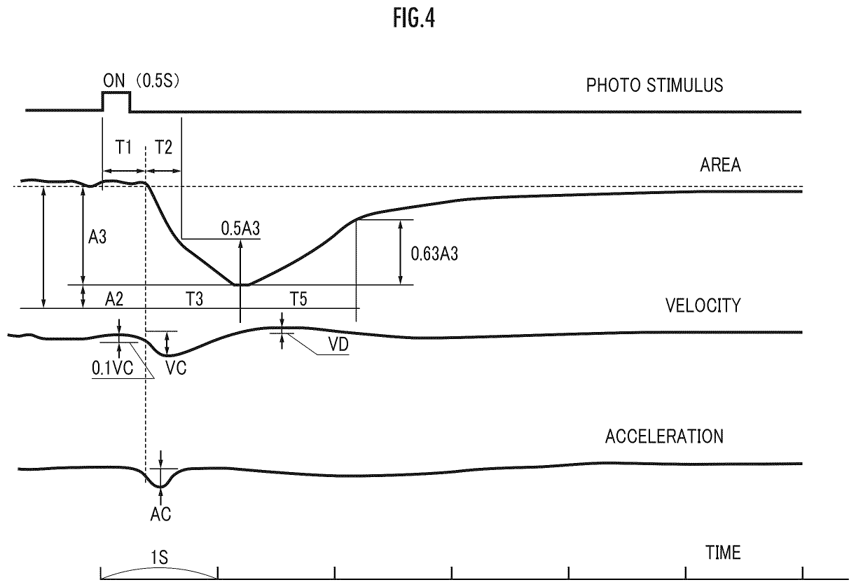

FIG. 4 is a diagram describing parameters (pupil indices) related to the pupil. In a case where the eye of the subject is irradiated with light, the size (area) of the pupil changes in a time-series manner as illustrated in the waveform of FIG. 4. The pupil dilation is realized by the action of the sympathetic nervous system, and the pupil constriction is realized by the action of the parasympathetic nervous system. In general, in a case where the sympathetic nervous system is dominant, the subject is in a tense and excited state, and in a case where the parasympathetic nervous system is dominant, the subject is in a relaxed and calm state. Therefore, by measuring the dilation and contraction of the pupil, the activity state of the sympathetic nervous system or the parasympathetic nervous system can be detected. FIG. 4 conceptually illustrates 12 parameters (pupil indices) related to the pupil: A1: pupil area in initial state [mm2], A2: minimum pupil constriction area after photo stimulus [mm2], A3: change pupil area after photo stimulus [mm2], CR: pupil constriction ratio (A3/A1) [%], D1: pupil diameter in the initial state [mm], T1: time from photo stimulus to the start of pupil constriction [msec], T2: time required for the change in change area to reach half [msec], T3: time required for the pupil to reach the minimum size thereof [msec], T5: time for the pupil to dilate from the minimum size and recover to 63% [msec], VC: maximum value of pupil constriction velocity [mm2/sec], VD: maximum value of pupil dilation velocity [mm2/sec], and AC: maximum value of pupil constriction acceleration [mm2/s2]. In general, it is considered that the sympathetic nervous function is reflected in VD and T5, and the parasympathetic nervous function is reflected in VC, CR, and A3.

In addition, from the waveform data of a test subject who responded in the subjective questionnaire with a feeling of very strong anxiety and tension, accompanied by chest tightness and difficulty breathing, it was found that the pupil constriction and pupil dilation velocities were reduced, and the pupil size did not readily return to its size immediately after light irradiation.

Furthermore, from the results of measuring the functional levels of the sympathetic nervous system or the parasympathetic nervous system for 424 test subjects (206 men and 218 women, average age: 45.4 years) and calculating the average values of the functional levels in each age group, it was found that there was a correlation between the maximum value (VD) of the pupil dilation velocity measured with the pupil meter and the neural function. Therefore, it is possible to calculate the age from the neural function level estimated based on the pupil indices such as the maximum value (VD) of the pupil dilation velocity. In this manner, the “neural age”, which is the age of the subject estimated from the pupil measurement, can be said to be an intuitively understandable index, as the “neural age” is a parameter that can also be compared with the actual age as information providing the neurological state.

The measurement time of one pupil measured by the pupil meter 10 is approximately 7 seconds, and the pupil index calculation unit 40 specifies the pupil for still images at each time point of a moving image in which the pupil of the subject is captured and obtains the pupil area to generate time-series data (waveform data) of the pupil area. In this case, there is a still image in which the pupil cannot be specified due to a cause such as blinking in the moving image data. In that case, the pupil index calculation unit 40 generates a waveform of the pupil area by linearly interpolating the pupil area obtained from the still image in which the pupil is normally specified before and after the time point at which the pupil cannot be specified. Further, the pupil index calculation unit 40 calculates the above-described 12 pupil indices.

As described above, although the waveform data of pupil area can be generated through interpolation even in the presence of blinking, when blinking occurs too frequently, there is a risk that the accuracy of pupil measurement may decrease. Therefore, in a case where the pupil index calculation unit 40 determines that the number of still images in which the pupil cannot be specified is larger than a predetermined threshold value, the pupil index calculation unit 40 transmits an instruction signal to display a message prompting the server 30 or the terminal device 50 to perform re-measurement.

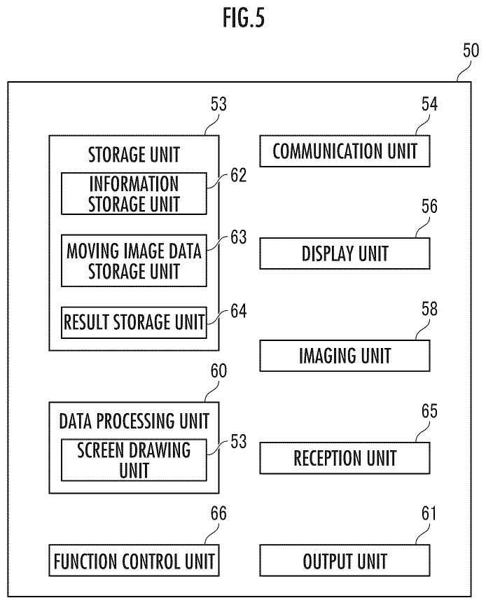

FIG. 5 is a diagram illustrating a functional configuration of the terminal device. The terminal device 50 includes: the communication unit 54 that receives data, such as an evaluation result or advice of a neurological state, which is an analysis result analyzed by the server 30, from the server 30 and transmits subject information to the server 30; and the display unit 56 that displays the evaluation result of the neurological state, a comment on the evaluation result, advice, and the like. The display unit 56 displays a plurality of pupil indices or neurological states at different time points or displays a change in the pupil index or the neurological state at different time points. For example, the display unit 56 displays a plurality of pupil indices or neurological states calculated from data obtained by measuring the pupil at different time points before and after the massage, or displays a change in the pupil index or neurological state before and after the massage. Therefore, the user or the subject of the present neurological state evaluation system can check, for example, the effect of the massage treatment from the visualized display information.

In addition, the terminal device 50 includes: a reception unit 65 that receives an input of subject information from the subject; the storage unit 53 that stores the input subject information, the neurological state estimation result, and the like; and a data processing unit 60 that processes data for drawing or function control. Further, an output unit 61 that outputs the stored information is provided.

The storage unit 53 includes: an information storage unit 62 that stores information such as text input by the subject; a moving image data storage unit 63 that stores moving image data; and a result storage unit 64 that stores waveform data of the pupil area, an evaluation result of the neurological state, advice, and the like.

The data processing unit 60 includes: a screen drawing unit 53 that displays the analysis result on the display unit 56; and a function control unit 66 that controls the function of the terminal device 50.

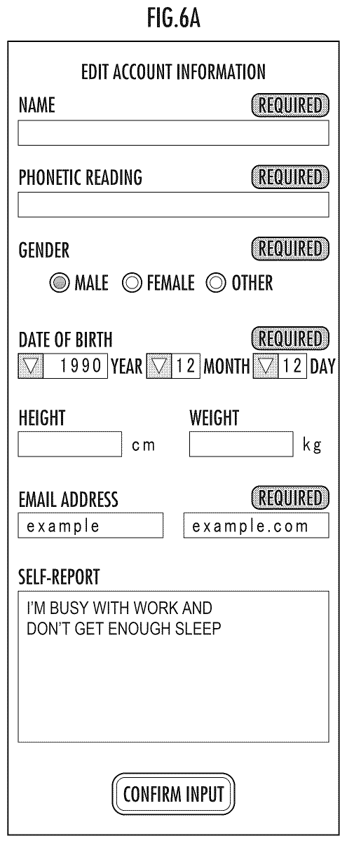

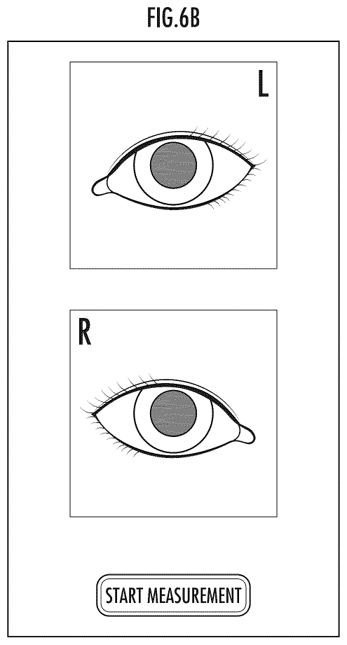

FIG. 6A is a diagram illustrating an input screen on a terminal device side of a neurological state evaluation system.

FIG. 6B is a diagram illustrating a screen for receiving a measurement start on the terminal device side of the neurological state evaluation system.

As illustrated in FIG. 6A, the input screen on the terminal device side of the neurological state evaluation system is provided with a field for entering the name of the subject, a check box for gender, a field for entering the date of birth, and a field for entering the height, weight, contact information, and a self-report. In a case where the input is completed, the input information is confirmed by touching the input confirmation button, and the screen transitions to the screen illustrated in FIG. 6B. In the screen illustrated in FIG. 6B, the videos of the left and right pupils of the subject are displayed. In a case where the subject determines that the pupil is positioned near the center of the image and is in a measurable state, the subject touches a button for starting measurement, and the measurement by the pupil meter 10 is started. However, the instruction to start measurement may be in a form in which the server 30 side issues an instruction to start measurement.

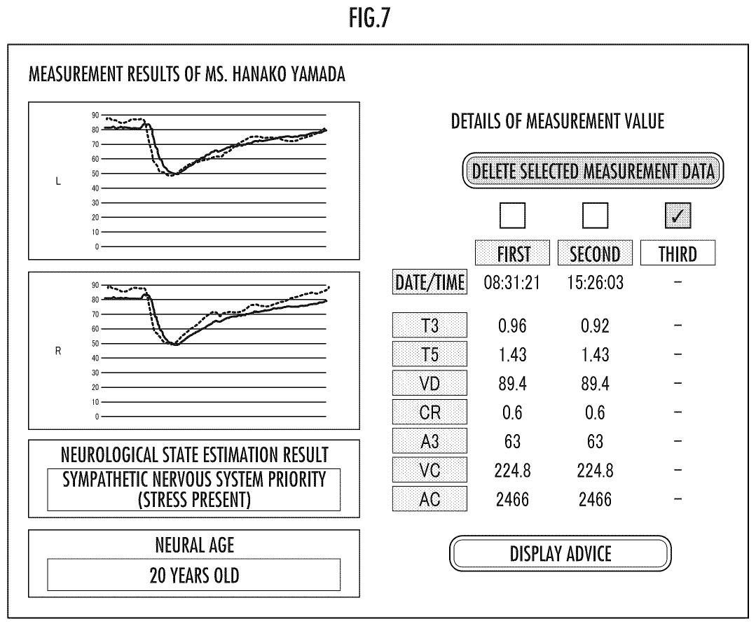

FIG. 7 is a diagram illustrating a measurement result display screen of the terminal device. A waveform indicating a change in pupil area, a numerical value of a main pupil index, a neurological state estimation result, and a neural age are displayed on the display screen. The waveform showing the change in pupil area and the numerical values of main pupil indices can be displayed for the plurality of measurement results. In particular, since the numerical value and the waveform of the pupil index at different time points are displayed on the same screen on the display screen, the user including the subject can grasp the change in the neurological state between the first measurement and the second measurement from the difference (change) in the plurality of measurement results.

For example, by measuring the pupil before and after the subject who is under stress receives the massage treatment, the subject or the massage provider can confirm the effect of the subject being able to relax due to the massage as the visualized information.

In addition, in a case of targeting an athlete, by performing measurements before and after a match, it is also possible to detect pain that the athlete is unaware of from the waveform of the pupil measurement. In a case where there are symptoms such as a sprain, a spike-shaped waveform may be repeatedly observed during the recovery of the pupil size, but the athlete himself or herself may not be aware of the pain, and the detection of symptoms that lead to a major failure is also effective.

“The plurality of pupil indices or the plurality of neurological states at different time points is displayed or change of pupil index or neurological state at different time points is displayed” according to the claims includes not only displaying the pupil indices or neurological states on the same screen but also displaying the pupil indices or neurological states at different time points on a plurality of screens in a case of screen transition to display the neurological states at different time points in a comparable manner.

In the display screen illustrated in FIG. 7, there is a check box in the display field of each measurement value, and the checked data can be deleted by touching a button for deleting the selected measurement data.

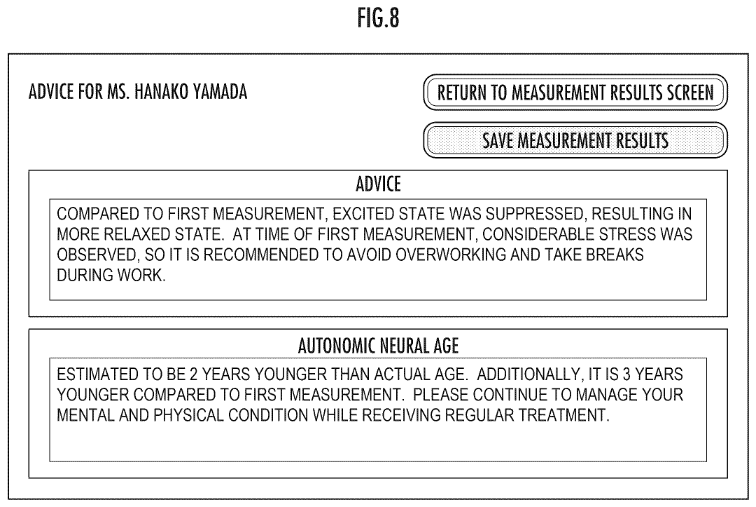

In addition, by touching the button for advice display, the screen on which the advice illustrated in FIG. 8 is displayed is transitioned.

FIG. 8 is a diagram illustrating an advice display screen of the terminal device. In the advice field, a comment describing the current neurological state, a comment regarding a change in the neurological state between the first time and the second time, and a comment regarding the improvement measure are displayed. In addition, in the comment field of the neural age, a comment on the neural age compared to the actual age or a comment on the change in the neural age due to the first measurement and the second measurement is described.

In addition, the measurement result can be transitioned to the screen on which the measurement result is displayed by touching a button for returning to the screen on which the measurement result is displayed, and the measurement data and the data of the estimation result can be stored in the storage unit 53 by touching a button for storing the measurement result.

### Processing of Neurological state Evaluation System

Next, processing of the neurological state evaluation system 1 will be described.

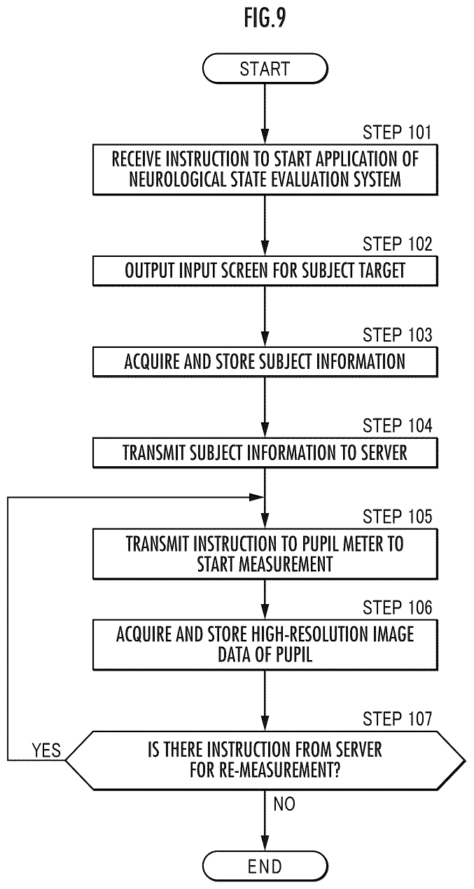

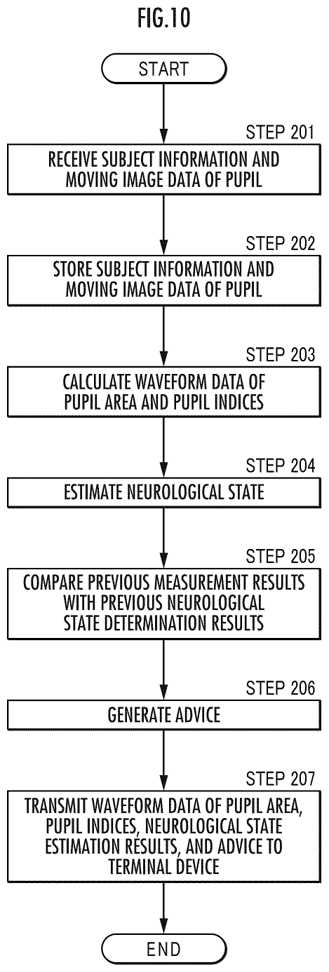

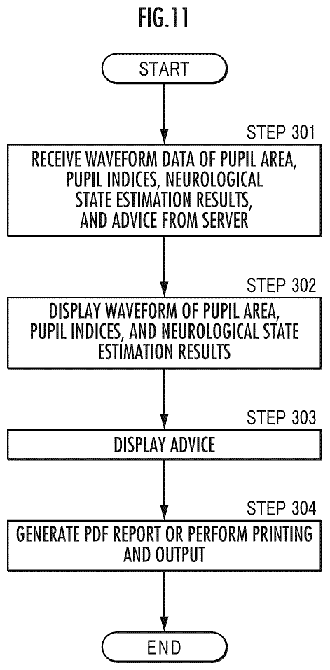

FIGS. 9 and 11 are flowcharts illustrating a flow of processing of the terminal device, and FIG. 10 is a flowchart illustrating a flow of processing of the server. FIG. 9 illustrates processing of transmitting the moving image data captured by the pupil meter 10 to the server 30 and the terminal device 50 and confirming whether or not the re-measurement is performed. FIG. 10 illustrates a flow of processing of evaluating the neurological state based on the moving image data of the pupil and the subject information transmitted from the pupil meter 10. FIG. 11 illustrates processing from when the terminal device receives the analysis results from the server 30 to when the analysis results are displayed.

### Processing of Terminal Device until Pupil Measurement is completed (First Processing)

As illustrated in FIG. 9, the function control unit 66 illustrated in FIG. 5 receives designation of the start from the subject and starts the application of the neurological state evaluation system (step 101). Next, the screen drawing unit 53 outputs the input screen illustrated in FIG. 6A (step 102). The subject inputs subject information such as the name, date of birth, height, weight, gender, and self-report of the subject on the input screen illustrated in FIG. 6A. The information storage unit 62 stores the input subject information (step 103). Next, the function control unit 66 transmits the subject information input via the communication unit 54 to the server 30 (step 104). When the measurement start button is touched, the function control unit 66 transmits an instruction to start measurement to the pupil meter 10 (step S105). The pupil meter 10 that has received the instruction to start measurement transmits the moving image data to the server 30 and the terminal device 50. The terminal device 50 acquires the moving image data from the pupil meter 10 or the server 30 and stores the moving image data in the moving image data storage unit 63 (step S106). Next, in a case where the terminal device 50 receives the measurement instruction signal from the server again, the terminal device 50 instructs the pupil meter 10 to start the measurement. Otherwise, the first processing of a terminal device 50 ends.

### Analysis Processing in Server

Next, processing of the server 30 will be described.

FIG. 10 is a flowchart illustrating a flow of processing of the server 30. The server 30 receives the moving image data of the pupil of the subject and the subject information from the pupil meter 10 or the terminal device 50 (step S201). The subject information storage unit 45 stores the subject information, and the analysis result storage unit 46 stores the moving image data (step S202). The pupil index calculation unit 40 calculates the waveform data of the pupil area and the pupil index from the received moving image data (step S203). The state estimation unit 41 estimates the neurological state from the pupil index (step S204). The comment generation unit 43 generates a comment from the estimated neurological state or the comparison between the previous measurement result and the current measurement result (step 205). The improvement measure generation unit 44 generates advice such as an improvement measure (step S206). Further, the communication unit 34 transmits the waveform of the pupil area, the pupil index, the estimation result of the neurological state, and the advice to the terminal device 50 (step S207).

### Processing of Terminal Device that Receives Estimation Result of Neurological state from Server

As illustrated in FIG. 11, the terminal device 50 receives the waveform data of the pupil area, the pupil index, the neurological state estimation result, and the advice from the server 30 (step S301). The screen drawing unit 53 of the terminal device 50 displays the waveform of the pupil area, the pupil index, and the estimation result of the neurological state received from the server 30 (step S302). Next, the screen drawing unit 53 displays the advice in response to the instruction of the screen transition by the subject (step S303), the subject gives an instruction to output, and accordingly, the output unit 61 generates the analysis report as a PDF file or prints and outputs the report (step S304).

As described above, the neurological state evaluation system 1 to which the present embodiment is applied can provide the evaluation result of the neurological state of the subject to the user or the subject by performing the action analysis on the moving image in which the pupil of the subject is captured.

The present embodiment has been described on the premise that the subject is the user of the neurological state evaluation system 1, but the user of the neurological state evaluation system 1 may be used separately from the subject. For example, the subject may be an employee of a company, and the user may be a person in a department that manages the health status of the employee or a doctor. The user may use the neurological state evaluation system 1 in a form of operating the server 30 or the terminal device 50.

In the present embodiment, the pupil meter 10, the server 30, and the terminal device 50 are separated, and each of the functions are described. However, the present invention is not limited to each of the functions being in any of the pupil meter 10, the server 30, or the terminal device 50. The neurological state evaluation system according to the present invention may be configured such that the pupil meter 10 or the terminal device 50 performs a part or all of the functions performed by the server 30. In addition, the pupil meter 10 and the terminal device 50 may function as an integrated device even in a case where the pupil meter 10 and the terminal device 50 are not connected to each other via the network 70. Further, in a case where the processing capacity and the storage capacity of the terminal device can be sufficiently secured, the system may be configured such that all the processing of the server 30 is executed by the terminal device 50.

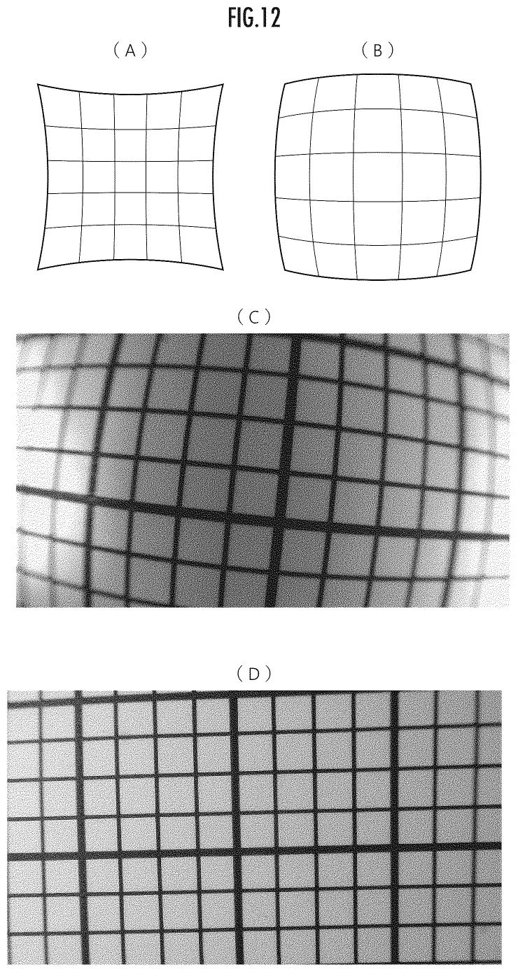

In the present embodiment, the imaging unit 17 can use a distortion-corrected lens having little distortion. In general, in a lens, distortion, blurriness, and the like occur due to the fact that light passing through the lens is not focused on one point. A phenomenon in which a shape on an object plane and a shape on an image plane are not similar to each other is called “distortion”, and appears as a phenomenon in which an image is distorted. As illustrated in FIG. 12(A), the case where the image contracts more toward the edges is referred to as barrel distortion. Meanwhile, as illustrated in FIG. 12(B), the case where the image is stretched more toward the edges is referred to as pincushion distortion. In the pupil measurement, due to distortion of the lens of the imaging unit 17, for example, a part of the image that is stretched has a large area on the image, and a part of the image that is compressed has a small area on the image. In a case where the pupil image is displayed and the area is to be obtained by counting the number of pixels corresponding to the pupil, the area of the part where the image is stretched is calculated excessively, and the area of the part where the image is compressed is calculated insufficiently. In other words, when distortion occurs in the image due to parts of the image being stretched or compressed, attempting to determine the area of the object displayed in the image by counting pixels results in large errors. That is, it is difficult to accurately calculate the pupil area from the image data having distortion. FIG. 12(C) illustrates an image in which an object that is originally a square lattice is distorted in a barrel shape. However, in a case where the mesh area of one square is counted in terms of the number of pixels and calculated, the mesh area of the peripheral portion is smaller than that of the central portion.

In the present embodiment, measurement is performed using a distortion-corrected lens with less distortion. Since the distortion-corrected lens has little distortion as illustrated in FIG. 12(D), the image forms an accurate similar shape of the real object. Therefore, the pupil area can be obtained based on the image captured by the distortion-corrected lens. Specifically, the pupil index calculation unit 40 calculates the pupil area by counting the number of pixels corresponding to the pupil based on the image data in which the pupil is captured with the distortion-corrected lens. The count value of the number of pixels does not calculate an absolute value of the area of the pupil, but represents a value obtained by converting the pupil area at a constant scale. Therefore, the rate of change of the value obtained by counting the pixels corresponding to the pupil from a pupil image captured with the distortion-corrected lens can be measured as the accurate rate of change of the pupil area.

In the related art, in a case of measurement with a distortion lens, it is necessary to set the center of the pupil to be positioned at the central portion with less distortion and perform capturing. A deviation in the position of the pupil in the captured image causes a calculation error of the pupil velocity or the pupil dilation velocity. However, as described above, by calculating the rate of change in the number of pixels corresponding to the pupil for the image of the pupil captured by the distortion-corrected lens, the maximum value (VD) of the pupil dilation velocity and the maximum value (VC) of the pupil constriction velocity can be accurately calculated.

In the present embodiment, the actual age and the neural age input by the subject can be compared to give advice. For example, in a case where the neural age is 5 years or more higher than the actual age, it is possible to convey to the subject, as a message, that they are in a state where concentration is lacking and performance is not improving. On the contrary, in a case where the neural age is 5 years or more lower than the actual age, a state of high concentration and improved performance is indicated.

In addition, in the present embodiment, the pupil index can be calculated from the left and right pupils of the subject, and the neural age of each of the left and right pupils can be calculated. In the case of a normal person, there is no large difference in the left and right neural ages. However, in a case where there is a concern about the function of the half body (for example, in a case where the right foot is painful), there may be a difference between the left and right sides. In a case where the difference between the left and right is deviated from the certain value or more, it is possible to give advice such as prompting an alert.

Since the pupil dilation is realized by the action of the sympathetic nervous system and pupil constriction, and the pupil constriction is realized by the action of the parasympathetic nervous system, the maximum value (VD) of the pupil dilation velocity and the maximum value (VC) of the pupil constriction velocity can be indices of the states of the sympathetic nervous system and the parasympathetic nervous system. Here, the “sympathetic neural age” is an age that represents to which age level of the neural function level the subject corresponds, based on a standard functional level by age, as in the simple neural age described above, but is an index calculated based on a pupil index controlled by the sympathetic nervous system. For example, the sympathetic neural age is calculated based on the maximum value (VD) of the pupil dilation velocity. In addition, in the present specification, the sympathetic neural age may be referred to as a neural age of the sympathetic nervous system.

Meanwhile, the “parasympathetic neural age” is an age that represents to which age level of the neural function level the subject corresponds, based on a standard functional level by age, as in the simple neural age described above, but is an index calculated based on a pupil index controlled by the parasympathetic nervous system.

For example, the parasympathetic neural age is calculated based on the maximum value (VC) of the pupil constriction velocity. In the present specification, the parasympathetic neural age may be referred to as a neural age of the parasympathetic nervous system.

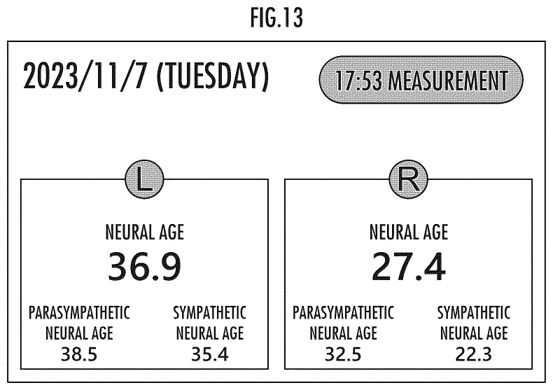

For example, FIG. 13 is a diagram illustrating a measurement result display screen of the terminal device. In the display drawing of the measurement result, the calculated neural age from the left pupil measurement is 36.9 years old, the calculated neural age from the right pupil measurement is 27.4 years old, the calculated sympathetic neural age from the left pupil measurement is 35.4 years old, the calculated sympathetic neural age from the right pupil measurement is 22.3 years old, the calculated parasympathetic neural age from the left pupil measurement is 38.5 years old, and the calculated parasympathetic neural age from the right pupil measurement is 32.5 years old.

From this measurement result, it can be seen that the sympathetic neural age of 22.3 years calculated from the right pupil measurement is 10 years or more younger than the sympathetic neural age of 35.4 years calculated from the left pupil measurement and the parasympathetic neural age of 32.5 years calculated from the right pupil measurement. By displaying the neural ages of the left and right sympathetic nervous systems and parasympathetic nervous systems in a comparative manner, it is possible to know whether or not there is an element lacking balance. In a case where the balance between the left and right sides and the balance between the sympathetic nervous system and the parasympathetic nervous system are lacking, the present invention device can issue an alert as data indicating a sign of some abnormality of the body or can display advice to the subject.

In FIG. 13, the neural ages of the left and right sympathetic nervous systems and the parasympathetic nervous systems based on the measurement result at a certain time point are displayed, but the neural ages of the left and right sympathetic nervous systems and the parasympathetic nervous systems at each of the measurement time points can also be displayed based on the measurement results at different time points. Further, it is also possible to display a change in a neural age of the left and right sympathetic nervous systems and/or parasympathetic nervous systems.

In a case where the neural age is evaluated as an average of measurement results of the left and right pupils or in a case where the neural age is evaluated without separating the sympathetic nervous systems and the parasympathetic nervous systems, it is difficult to notice the abnormality even in a case where there is an abnormality in either the left or right side or in either the sympathetic nervous systems or the parasympathetic nervous systems. By displaying either the left or right side or both the neural ages of the sympathetic nervous systems or the parasympathetic nervous systems, it becomes possible to detect abnormalities in the neurological state of the subject, for example, by detecting an abnormality in the neural age of the left parasympathetic nervous system. In this manner, not only the simple presentation of neural age based on measurement at a certain point in time, but also the display of the neural ages of each of the left and right sympathetic nervous systems and the parasympathetic nervous systems and their temporal changes enables more detailed detection of the neurological state of the subject.

### DESCRIPTION OF REFERENCE NUMERALS

1 . . . neurological state evaluation system
10. . . . pupil meter
11 . . . control unit
12 . . . memory
13 . . . storage unit
14 . . . communication unit
15 . . . operation unit
16 . . . light emitting unit
16a . . . visible light emitting unit
16b . . . infrared light emitting unit
17. . . imaging unit
30 . . . server
31 . . . control unit
32 . . . memory
33 . . . storage unit
34 . . . communication unit
35 . . . operation unit
36 . . . display unit
37 . . . display control unit
38 . . . reception unit
39 . . . data processing unit
40 . . . pupil index calculation unit
41 . . . state estimation unit
42 . . . model generation unit
43 . . . comment generation unit
44 . . . improvement measure generation unit
45 . . . subject information storage unit
46 . . . analysis result storage unit
47 . . . training-related data storage unit
48 . . . moving image data acquisition unit
49 . . . analysis result output unit
50 . . . terminal device
51 . . . control unit
52 . . . memory
53 . . . storage unit
54 . . . communication unit
55 . . . operation unit
56 . . . display unit
57 . . . display control unit
58 . . . imaging unit
60 . . . data processing unit
61 . . . output unit
62 . . . information storage unit
63 . . . moving image data storage unit
64 . . . result storage unit
65 . . . reception unit
66 . . . function control unit
70 . . . network

1. A neurological state evaluation system comprising:
a light irradiation unit configured to irradiate a pupil of a subject with light;
an acquisition unit configured to acquire moving image data in which the pupil of the subject is captured;
a calculation unit configured to calculate a pupil index from the moving image data;
a state evaluation unit configured to evaluate a sympathetic neural age and a parasympathetic neural age of the subject based on the pupil index; and
a display unit configured to display the sympathetic neural age and the parasympathetic neural age.

2. The neurological state evaluation system according to claim 1, wherein the state evaluation unit evaluates left and right sympathetic neural ages and parasympathetic neural ages based on left and right pupil indices of the subject, and the display unit displays each of the left and right sympathetic neural ages and parasympathetic neural ages.

3. The neurological state evaluation system according to claim 1, wherein the acquisition unit acquires the moving image data captured by a distortion-corrected lens, and
the calculation unit calculates the pupil index by counting pixels related to the pupil in the captured moving image data.

4. The neurological state evaluation system according to claim 1, wherein the display unit displays a plurality of the sympathetic neural ages and the parasympathetic neural ages at different time points or displays a change in the sympathetic neural age or the parasympathetic neural age at different time points.

5. The neurological state evaluation system according to any one of claims 1 to 4, further comprising:
an advice generation unit configured to generate advice to the subject based on the sympathetic neural age and the parasympathetic neural age,
wherein the display unit displays the advice.
